(19) United States
(12) Patent Application Publication
(10) Pub. No.: US 2010/0196268 A1
(43) Pub. Date: Aug. 5, 2010

(54) Compositions and methods for treatment of tumors by direct administration of a radioisotope

(75) Inventors: R. Keith Frank, Lake Jackson, TX (US); Kenneth McMillan, (US); Jaime Simon, Angleton, TX (US); H.Max Loy, JR., Houston, TX (US); Stanley D. Stearns, Houston, TX (US); Edna Sue McMillan, Richwood, TX (US)

Correspondence Address: TECHNOLOGY LAW, PLLC, 3595 N. SUNSET WAY, SANFORD, MI, 48657, US

(73) Assignees: IsoTherapeutics Group LLC, Angleton, TX (US); Gabriel Institute, Inc., Houston, TX (US)

(21) Appl. No.: 12/798,473

(22) Filed: Apr. 5, 2010

(30) Foreign Application Priority Data
Feb. 15, 2008 (US) PCT/US2008/002026

(51) Int. Cl. A61K 51/00; A61P 35/00

(52) U.S. Cl. 424/1.61

(57) ABSTRACT

This invention provides a safer and more effective treatment for non-intracavitary undesirable tissue masses, especially bone cancer and soft tissue tumors. The method involves the direct administration of a therapeutically-effective dose of a formulated radioisotope composition nearby or directly into the tissue mass. Small volumes of the composition are used.

Administration of the dose for bone cancer may be done through a hole or multiple holes created in the bone using a miniature drill. Delivery of the dose directly into a tumor may be accomplished using a microsyringe or a miniature pump capable of accurately delivering microliter amounts of material.

### CROSS REFERENCE TO RELATED APPLICATIONS

This application claims the benefit of priority from international application PCT/US2008/002026, filed Feb. 15, 2008, which claims the benefit of U.S. Provisional Patent Applications 60/997,856 and 60/997,873, both filed on Oct. 5, 2007.

This application is related to US Published Patent Application 20090228014, published Sep. 10, 2009, filed on May 8, 2009 under U.S. Ser. No. 12/437,910 and WO2008/103,606, published 28 Aug. 2008.

### FIELD OF THE INVENTION

The present invention concerns treatment of undesirable tissue masses, such as bone cancer or soft tissue tumors, in mammals and humans by administration of a radioisotope formulation directly to the area of the undesired tissue mass, i.e., via intratumural, intramedullary or intraosseous injection.

### BACKGROUND OF THE INVENTION

The treatment of cancerous tumors or masses of undesirable tissue has been of concern for many years with various attempts to have effective treatment to prolong the quality of life of the mammal or human. Various compositions have been tried and the following discussion of bone tumor and soft tissue tumor are discussed below.

Bone Cancer

According to the American Academy of Orthopaedic Surgeons, “More than 1.2 million new cancer cases are diagnosed each year [in the US], and approximately 50 percent of these tumors can spread or metastasize to the skeleton.” Metastatic bone cancer therefore afflicts over 500,000 patients in the US alone. Bone is the third most common site of metastatic disease. Cancers most likely to metastasize to bone include breast, lung, prostate, thyroid and kidney. In many cases there are multiple bone metastatic sites making treatment more difficult. Pain, pathological fractures and hypercalcemia are the major source of morbidity associated with bone metastasis. Pain is the most common symptom found in 70% of patients.

Primary bone cancer is much less prevalent (2,370 new cases and 1,330 deaths estimated in the US for 2007), but it is much more aggressive. This type of cancer is more likely to occur in young patients. In contrast to people, primary bone cancer is more prevalent in dogs than metastatic bone cancer. Large dogs frequently present with primary bone cancer. Because of the aggressive nature of the disease, primary bone cancer is often treated by amputation of the area affected to prevent the cancer from spreading. In addition, chemotherapeutic agents are then used to decrease the chance of metastatic disease, especially to the lungs.

The pain associated with bone cancer, especially metastatic bone cancer, is often treated with narcotics. However, the patients have need for increasing amounts of narcotics to control the pain. The side effects of the narcotics result in a significant decrease in the patient's quality of life.

Another method for treatment is external beam radiation or more recently stereotactic radiotherapy of bone metastatic sites. However, current treatments with high energy electromagnetic radiation do not exclusively deliver radiation to the tumor. This treatment results in the necessity to administer the dose over about a week and has the difficultly of giving high doses of radiation to a tumor without significant damage to surrounding tissue.

Intraoperative Radiation Therapy (IORT) has permitted localized tumor destruction, but this is expensive and associated with significant trauma due to surgery.

The ability to target bone tumors has been exploited in the field of radiopharmaceuticals for many years. Both diagnostic and therapeutic radiopharmaceuticals capable of targeting bone tumors generally use phosphonic acid functionality as the targeting moiety. For example, pyrophosphates have been used to deliver Tc-99m, a gamma-emitting diagnostic radioisotope, to bone. This technology was displaced by the bisphosphonates because of their increased stability in vivo. In addition, therapeutic radiopharmaceuticals for bone tumors were developed in the 1980's and 1990's. Of these, a series of chelates based on aminomethylenephosphonic acids offer another type of functionality useful for targeting bone tumors. Thus ethylenediaminetetramethylenephosphonic acid (EDTMP) has been shown to be a very good chelating agent for delivering metals such as Sm, Gd, Ho, and Y to the bone.

Two radiopharmaceuticals, both based on radioactive metals, are marketed in the United States for the treatment of bone metastases. Metastron® is an injectable solution of strontium-89 (Sr-89) given as the chloride salt. Quadramet® is a phosphonic acid (EDTMP) chelate of samarium-153 (Sm-153). Both of these agents concentrate in normal bone as well as in the metastatic lesions. This gives a radiation dose to the bone marrow resulting in temporary but significant suppression of the immune system. For that reason these agents are contraindicated when chemotherapeutic agents are planned. Thus a patient may suffer from bone pain while waiting to receive a chemotherapeutic regimen for the primary cancer.

When these available chelates are injected intravenously, about 50% of the injected dose concentrates in the bone. The rest is efficiently cleared by the kidneys and into the bladder; however, because of this clearance, toxicity to these organs has been observed when administering large therapeutic doses of bone seeking radiopharmaceuticals. The amount of radioactive metal deposited at the site of a bone tumor is significantly higher than in normal bone. Although the chelate concentration in the site of a tumor is as much as 20 times that of normal bone, significant amounts of radioactivity are taken up by normal bone. The dose from the bone to the bone marrow can suppress bone marrow. Even though this effect is usually temporary and marrow cells recover, the use of these agents are contraindicated when used with chemotherapeutic agents that also suppress bone marrow. Therefore therapeutic bone agents are typically not used at the same time chemotherapeutic agents are used. In addition, only a small fraction of the radiation dose is associated with the tumor. Because of the fast kidney clearance and uptake in normal bone, only about 0.1% of the dose goes to the site of the tumor. Administration of larger doses of bone agents is limited by the dose to the bone marrow.

An example of the bisphosphonate chelant, methylenediphosphonic acid (MDP), is shown in the structure below.

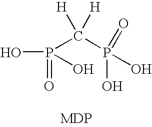

Two aminomethylenephosphonic acid chelants, ethylenediaminetetramethylenephosphonic acid (EDTMP) and 1,4,7,10-tetraazacyclododecane-1,4,7,10-tetra(methylenephosphonic acid) (DOTMP), are shown in the structures below.

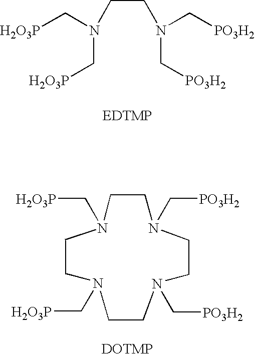

To date even combinations of treatments have not been effective at resolving bone tumors. Thus it is still common practice to amputate a limb to stop the spread of bone cancers. In the case of metastatic bone cancer, pain palliation and maintaining quality of life is often the goal in contrast to resolution of the tumors. There clearly is a need for more effective therapy to treat bone cancer.

Brachytherapy

In contrast to external beam radiotherapy, where an external beam of radiation is directed to the treatment area (such as discussed above for bone tumors), brachytherapy is a form of radiotherapy where a radioactive source is placed inside or next to the area requiring treatment. Brachytherapy is also known as sealed source radiotherapy or endocurietherapy and is commonly used to treat localized prostate cancer and cancers of the head and neck. Superficial tumors can be treated by placing sources close to the skin. Interstitial brachytherapy is where the radioactive source is inserted into tissue. Intracavitary brachytherapy involves placing the source in a pre-existing body cavity. Intravascular brachytherapy places a catheter with the source inside blood vessels.

In most of these cases the radioactive material is encapsulated in a metal casing. Because of this casing, most of the radioactive sources are electromagnetic radiation (X-rays and gamma photons) emitting radionuclides such that the radiation can penetrate the outer casing and deliver a radiation dose to surrounding tissue. Administration of the radioisotope without this encapsulation may result in migration of the radioisotope to other areas of the body creating side effects in the patient.

Particle emitting radionuclides such as beta (β) and alpha (α) emitters are rarely used in this application because a significant portion of the dose would not penetrate the casing within which the isotope is contained. However, in many cases the gamma photons penetrate beyond the desired treatment area resulting in significant side effects. Therefore, a more specific method to deliver radiation is needed.

The prostate is a gland in the male reproductive system located just below the urinary bladder and in front of the rectum. It is about the size of a walnut and surrounds the urethra. In 2007 the American Cancer Society estimated 218,890 new cases and 27,050 deaths due to prostate cancer in the US. Treatment options include surgery, external radiation therapy, and brachytherapy. In many cases brachytherapy is the preferred choice due to less trauma to surrounding tissues. However since the radioisotopes selected for this application are gamma (γ) emitters, delivering an undesired radiation dose to surrounding tissue remains a problem.

The radioactive sources used for brachytherapy are sealed in “seeds” or wires. Permanent prostate brachytherapy involves implanting between 60 and 120 rice-sized radioactive seeds into the prostate. One type of radioactive seed is based on 1-125 which has a 59.4 day half life and emits multiple X-rays around 30 keV. Recently a shorter half life alternative has been proposed with Cs-131 which has a 9.7 day half life and emits X-rays of about 30 keV. Alternatively, Pd-103 is used which has a 17 day half life and emits X-rays of about 20 keV. Another option is Ir-192 which has a half life of 73.8 days and gamma emissions at 468 keV. Ir-192 can be used to give different doses to different parts of the prostate. All these isotopes emit electromagnetic radiation that penetrates beyond the prostate and into normal tissue causing problems such as impotence, urinary problems, and bowel problems. Although in most cases the seeds stay in place, seed migration does occur in a portion of patients. Usually the seeds migrate to the urethra or bladder.

In some cases, brachytherapy is used to destroy cancer cells left over after a surgical procedure. For example breast cancer patients can be treated with a technology by the name of MammoSite® Radiation Therapy System. This involves a balloon catheter that is inserted into the area of the breast where a tumor was removed. The balloon is expanded and radiation is delivered via a small bead attached to a wire. Similarly, the space surrounding a resected brain tumor can be treated using a balloon catheter inflated with a radioactive solution of I-125. This technology is called GliaSite® Radiation Therapy System (e.g. U.S. Pat. No. 6,315,979). In these cases the balloon prevents the radioactivity from going systemic. Again, the radioisotopes used are those emitting penetrating electromagnetic radiation such as X-rays or gamma rays.

Beta emitting radioisotopes are being used in what could be categorized as brachytherapy. For example, liver cancer has been treated with a form of brachytherapy. This technology called Selective Internal Radiation Therapy (SIRT) delivers radioactive particles to a tumor via the blood supply. The radioactive particles are positioned via a catheter in the hepatic artery, the portal vein, or a branch of either of these vessels. The catheter is guided to the branch of the blood vessel that feeds the tumor, and then the microspheres are infused. The radioactive microspheres become trapped in the capillary beds of the tumor and the surrounding tissues which results in a more targeted radiation dose to the tumor. There are currently two products that take this approach, both are microspheres labeled with Y-90, TheraSphere® (MDS Nordion, Inc.), and SIR-Spheres® (SIRTeX® Medical). TheraSpheres are glass microspheres which have a diameter of 25±10 μm so they are trapped mainly within tumor terminal arterioles, which are estimated to have a diameter of 8-10 μm. SIR-Spheres are resin-based microspheres that are approximately 32 μm in diameter. One issue with both of these products is that a portion of the radioactive microspheres can migrate to other tissues such as the lungs and cause undesired side effects.

Ho-166 bound to chitosan has also been proposed to treat cancer cells. Thus J. Nucl. Med. 39(12), 2161-6 (1988 December) describes a method to treat liver cancer by administering the compound via the hepatic artery. However, “shunting” of radioactivity to the lung has again been a problem. In addition, it is a cumbersome technique to determine the blood supply to the tumor and to deliver the particles in the selected blood vessels.

U.S. Pat. No. 5,320,824 describes the use of rare earth isotopes such as Sm-153 and Ho-166 bound to hydroxyapatite for the treatment of rheumatoid arthritis. In this process, most of the radioisotope bound to hydroxyapatite either remains in the injected joint or is taken up by the synovial membrane surrounding the joint.

Localization to the target tissue depends on phagocytosis of the hydroxyapatite particles into the synovial membrane. One major problem with this approach is leakage of radioisotope from the synovial cavity to other parts of the body.

Kyker et al., Federation Proc. 13, 245-246 (1954), Lewin, et al., J. Nat. Cancer Inst. 15, 131-143 (1954), and Andrews et al., International Conference on the Peaceful Uses of Atomic Energy, Vol. 10 pp 122 (1956), describe attempts to treat cancer by forming radioactive colloids in situ in the body with limited success.

As is evident from the discussion above, better technology to ablate undesirable cells is needed. In the field of brachytherapy, more effective methods of delivering radioisotopes to tumors are needed that give a radiation dose specifically to the treatment area with little to no dose to non-target tissues.

### SUMMARY OF THE INVENTION

An aim of this invention is to provide a pharmaceutically-acceptable composition and therapeutic method that can deliver relatively large radiation doses from a radioisotope in a minimal volume to the site of an undesired tissue mass, including infections and cancerous tumors in both soft tissue and bone, for the purpose of killing said undesirable tissue. A further aim of this invention is to minimize the amount of radiation dose to non-target tissues in order to minimize side effects.

One aspect of this invention concerns a composition and a method for the therapeutic treatment of a non-intracavitary undesirable tissue mass in an animal or human in need of such treatment. More specifically, an embodiment of this invention provides a pharmaceutically-acceptable composition which comprises insoluble particles in a pharmaceutically-acceptable, aqueous medium, obtained by adding an alkaline material to an aqueous solution comprising a rare earth or rare earth-type radionuclide or combination thereof, forming a suspension, slurry or emulsion, said composition having a pH greater than about 7 and wherein a therapeutically-effective quantity thereof is administered in one or more locations into or near a non-intracavitary undesirable tissue mass in one or more locations in an animal or human such that greater than about 75% of the administered quantity remains at the site of administration for at least two half lives of the radionuclide.

Administration of the therapeutically-effective dose is accomplished by the direct administration of a very small volume of a formulation to the desired site. The radioactivity delivered to the site remains at the site of administration for a sufficient time to give a therapeutic radiation dose to that area. Compared to systemic administration approaches, the total amount of radioactivity administered is very small and the amount of radioisotope that leaches out of the treatment area is minimal, thus little to no radiation dose to normal tissues is realized.

Administration of the radioisotope formulation can be via a microsyringe or another device capable of delivering small volumes of fluid such as a small pump. In one embodiment of the invention for treating bone tumors, a miniature drill is used to create one or more holes by which a catheter can be inserted through the holes and a device capable of delivering small volumes of fluid is used to deliver the dose. In other embodiments, a microsyringe can be used for delivery.

This invention concerns a better therapeutic approach to the treatment of cancer by the administration of a very small volume of therapeutic radioisotope directly to the tissue to be treated. Radioisotopes of this invention include particle-emitting isotopes such as alpha (α) emitters or beta (β) emitters that can deposit therapeutic amounts of ionizing radiation at the site of the tissue.

### BRIEF DESCRIPTION OF THE DRAWINGS

FIG. 1 shows the results obtained graphically for Example B and Example 2 of tumor size vs. time for treated and control mice.

FIG. 2 is a copy of an X-ray showing osteosarcoma in the left proximal radius of a female Great Dane as treated in Example 7.

FIG. 3 is a copy of a photograph showing the custom-made three piece adapters (hypodermic, cortex, stylet) and micro bone drill (Valco Instruments Company) as used in the procedure of Example 7.

FIG. 4 is a copy of an X-ray showing osteosarcoma in the left distal radius of a female Saint Bernard as treated in Example 8.

### DETAILED DESCRIPTION OF THE INVENTION

### Glossary

The following terms as used in this application are to be defined as stated below and for these terms, the singular includes the plural.
CT means computed tomography, usually X-ray computed tomography
Intracavitary means a pre-existing body cavity such as a sinus cavity; non-intracavitary means not in a pre-existing body cavity
MRI means magnetic resonance imaging
PET means positron emission tomography

This invention involves the delivery of a therapeutically-effective amount of a pharmaceutically-acceptable formulated radioisotope composition directly to an undesired tissue mass, including infections (e.g., osteomyelitis) and cancerous tumors, especially inoperable cancerous tumors, in both soft tissue and bone, such as cancerous tumors in bone, prostate, liver, lung, brain, muscle, breast, cervix and skin. These tumors are considered as occurring in non-intracavitary body areas. Because the amount administered to the animal, including humans, of these formulations is very small in volume and the amount of radioactivity administered is effectively directed to the desired site, the administration is not by means that involve other body areas, e.g., no systemic administration (such as I.V. administration) is intended. Non-target, normal tissue is spared because only a very small amount of radioisotope is administered and the majority of the radioisotope mixture is immobilized at the administration site. Thus the majority of the radioactive decay of the isotope occurs at the site of injection with only small amounts of radioactivity leaching out of the injection site before a significant amount of the radioisotope decays. This results in a high radiation dose to the target area and extremely small doses to non-target tissues. The composition can be used to treat a variety of conditions, particularly cancerous tumors.

Radioisotopes used in this invention are particle emitters (beta (β) emitters or alpha (α) emitters). Preferred radioisotopes are ions of rare earth metals and rare earth-type metals including Pm, Sm, Gd, Dy, Ho, Yb, Lu, and Y; especially preferred are Sm, Ho, Lu, and Y. Preferred radioactive isotopes include: Sm-153, Ho-166, Y-90, Pm-149, Gd-159, Lu-177, Yb-175, Pb-212, Bi-212, Bi-213, and Ac-225. Especially preferred are Sm-153, Ho-166, Y-90, Bi-212, Bi-213, Ac-225, and Lu-177. Most preferred are isotopes with a relatively short half life of less than about 3 days that emit energetic beta particles. Examples of such isotopes include Y-90, Ho-166, and Sm-153. It is understood that often the radioisotopes contain non-radioactive carrier isotopes as a mixtures.

In one aspect of this invention, insoluble radioactive particles are prepared by adding an alkaline material (e.g. NaOH or KOH) to an aqueous solution comprising the rare earth or rare earth-type radionuclide(s) to obtain a pH wherein insoluble particles are formed, which usually results in forming a suspension, slurry or an emulsion. This desired pH often varies from metal to metal. Preferred pH for precipitation of the particles is usually greater than about 7. A more preferred pH is greater than about 8. A pH range from about 8 to about 14 is preferred for most of the radioactive metals, and a more preferred pH range is from about 8 to about 11. The desired pH is obtained by the addition of a suitable base such as sodium or potassium hydroxide to the aqueous radioisotope. Once formed, the radioactive particles can be administered in a therapeutically-acceptable dose and in a pharmaceutically-acceptable medium such as water or saline.

The formulated composition may be a suspension, a slurry or an emulsion. Optionally, other usual pharmaceutically-acceptable ingredients can be present in the composition such as excipients, suspension aids, preservatives, buffers for pH adjustment, and others, which are well known to one skilled in this art.

In another aspect of this invention, the composition containing the insoluble particles are then separated from the initial composition (e.g. by filtering, centrifuging, or decanting) and a therapeutically-effective quantity of the insoluble particles is administered in a pharmaceutically-acceptable medium into or near the undesirable tissue mass and wherein greater than about 75% of the administered dose remains at the site of administration for at least two half lives of the radionuclide(s).

In yet another aspect of this invention, the amount of the radioisotope administered is very low. Preferred volumes of administered therapeutic radiation doses of radioisotope in the present composition are less than about 50 microliters per cubic centimeter of undesirable tissue mass (50 μL/cm³). More preferred are volumes of less than 20 microliters per cubic centimeter of undesirable tissue mass (20 μL/cm³). Even more preferred are volumes of less than 10 microliters per cubic centimeter of undesirable tissue mass (10 μL/cm³). Most preferred is about 2 microliters per 0.5 cubic centimeter of undesirable tissue mass. Delivery of the formulated composition can be done using a microsyringe or a pump capable of accurately delivering microliter volumes (e.g. Valco Instrument Company, Inc. model CP-DSM) to provide flow to the proximal end of a catheter which may be placed within or next to the undesirable tissue mass to be treated. The flow may be either continuous or may be pulsed to enhance complete penetration of the undesirable tissue mass by the radioisotope.

Therapeutically-effective doses of radioisotopes will require different amounts of activity for different isotopes but can be described by the radiation dose delivered to the tissue. Preferred doses are at least about 20 Gy. More preferred is a dose of at least about 50 Gy.

In one embodiment of the invention, the radioisotope may be delivered to a bone tumor using a miniature pump or syringe. Access to the tumor may be effected by the use of a bone biopsy tool or a miniature drill capable of making a curved or angled hole through bone and either upstream of the tumor (so to guide the catheter towards it) or directly into the bone or tumor in the bone. The insertion of the catheter using imaging techniques, as is known in the art, may help to position the distal end of the catheter in close proximity to the tumor. Some known imaging techniques for this use are PET, CT, Ultrasound, MRI, and fluoroscopy; particularly useful are PET or CT.

The drill used in the present examples is discussed in U.S. Provisional Patent Applications 60/890,831, filed on Feb. 20, 2007 and 60/891,183, filed on Feb. 22, 2007, now US Published Patent Application 20090228014, published Sep. 10, 2009, and WO2008/103,606, published 28 Aug. 2008, but this invention is not limited to the use of this drill as any device that can provide a suitable hole in the bone, such as a syringe needle or biopsy tool will suffice.

This invention will be further clarified by a consideration of the following examples, which are intended to be purely exemplary of the present invention. The following numbered examples illustrate this invention; and the lettered examples are illustrating comparative examples.

### EXAMPLES

### General Information

All percentages are weight/weight (w/w) unless stated otherwise.

MURR is University of Missouri Research Reactor (Columbia, Mo.) that has a service to provide radioisotopes.

It is understood that often the radioisotopes contain non-radioactive carrier isotopes as mixtures.

### Example 1

### High pH Lu-177 Composition

A composition produced at high pH was prepared by adding of 2.0 μL of 50% w/w NaOH to 10 μL of a Lu-177 solution (obtained from MURR, 1.09 Ci/mL in 0.05 M HCl) followed by the addition of 8.0 μL of water. The mixture was allowed to stand for 30 minutes prior to injection. The pH of the composition was greater than about 10.

### Example A (Comparative)

### Low pH Lu-177 Solution

A solution of Lu-177 in 0.05 M HCl was obtained from MURR containing about 1.09 Ci/mL. The injectate was prepared by mixing equal volumes of the Lu-177 solution and 0.05 M HCl. The pH was less than about 2.

### Example 2

### In Vivo Xenograft Test High pH Lu-177

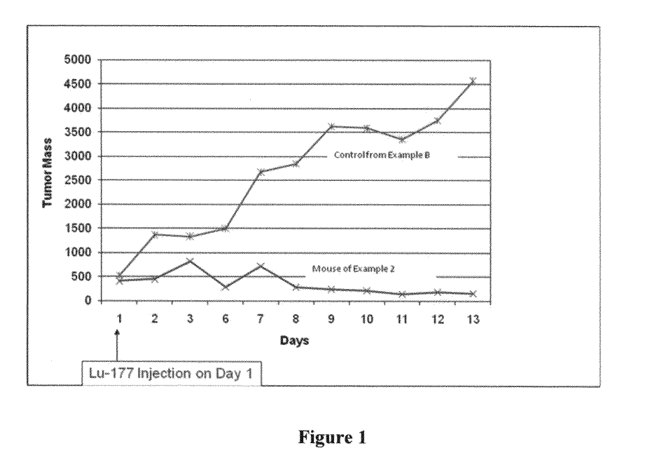

An athymic mouse bearing an HT-29 xenograft was anesthetized and 2-3 μL of the composition of Example 1 was diluted with about 20 μL of water and administered directly into the tumor. Multiple injections were made at several different sites around the periphery of the tumor as well as directly into the tumor mass. The amount of injected activity was determined to be 0.924 mCi Lu-177. Gamma camera images 13 days post-treatment showed the majority of the activity remaining at the injection site. Less than 1 μCi of the Lu-177 was found in the urine or feces on any of the 13 days post-injection. The size of the tumor was measured and compared to a similar mouse injected with saline as a control. The tumor in the saline control mouse increased in size while the tumor in the mouse of this Example 2 decreased in size. These results are shown in Table 1 below and graphically in FIG. 1.

TABLE 1
Tumor size in cubic millimeters for treated and control mice

| | Days Post Treatment | | | | | | | | | | |
|---|---|---|---|---|---|---|---|---|---|---|---|
| | 1 | 2 | 3 | 6 | 7 | 8 | 9 | 10 | 11 | 12 | 13 |
| Mouse of | 405 | 447 | 816 | 282 | 721 | 282 | 237 | 211 | 144 | 181 | 154 |
| Example 2 | | | | | | | | | | | |
| Control | 527 | 1362 | 1332 | 1499 | 2673 | 2847 | 3621 | 3589 | 3357 | 3748 | 4573 |
| Mouse | | | | | | | | | | | |
| Example B | | | | | | | | | | | |

### Example B (Comparative)

### In Vivo Xenograft Test Low pH Lu-177

An athymic mouse bearing an HT-29 (human colorectal carcinoma) xenograft was anesthetized and 2-3 μL of the solution of Example A was administered directly into the tumor. The amount of injected activity was determined using a dose calibrator to be 1.08 mCi of Lu-177.

The fate of the Lu-177 in the mouse body was determined using a gamma camera. In addition, a dose calibrator was used to measure the amount of radioactivity collected in the urine as a function of time. After 1 day, 50 μCi of Lu-177 was found in the collected urine and feces. In addition, significant migration of radioisotope from the tumor area was observed in the gamma camera images. Over time, the mouse showed signs of increasing morbidity. The mouse was euthanized due to morbidity after a 20% loss in body weight 9 days post-injection.

### Example 3

### In Vivo Prostate Test

A volume of about 6-8 μL of the composition of Example 1 was administered to the left lobe of the prostate of a normal Sprague Dawley rat while the rat was under anesthesia. The rat received a Lu-177 dose of 0.924 mCi.

The rat was monitored daily for Lu-177 in urine and feces. Only minimally measurable Lu-177 (−1.0 μCi) was found on any individual day. Gamma images showed the Lu-177 remaining at the injection site throughout the 7 day study with very little systemic radioactivity. The rat was euthanized seven days post-treatment, organs and tissues were excised and the presence of Lu-177 in each was determined. Less than 10% of the dose was found outside the prostate after 7 days. Examination of the prostate revealed the injected lobe of the prostate to be atrophied compared to the opposite lobe of the prostate.

### Example 4

### Lu-177 Injectate Preparation

Lu-177 was received from MURR in 0.1 M HCl at 0.71 mCi/μL upon arrival. Activity was measured using a Capintec™ CRC-15 dose calibrator. To 3.0 μL of this solution was added 3.0 μL of 1.0 N NaOH (Fisher) to form the composition. Water was added to give a final volume of the composition of 10.0 μL.

### Example 5

### Lung Test—Lu-177 Injection into a Male Sprague Dawley Rat

A 364 g male Sprague Dawley rat, under anesthesia, was injected with 3-5 μL (˜1.0 mCi) of the composition in Example 4 directly into the lung using an insulin syringe. The dose was deposited in the left lobe of the lung via needle insertion through the skin.

Images of the rat using a gamma camera were taken at 30 minutes post injection, at 18 hours, and at 2, 5, 7 and 9 days post injection. Feces and urine excretions were collected daily and analyzed for the presence of radioactivity. At 9 days the rat was euthanized and organs/tissues obtained for gamma counting.

All gamma images showed one single spot at the site of injection with no detectable activity in any other part of the body.

Gamma counting of low activity tissues was accomplished using a Wizard™ 1480 gamma counter (Packard); highest activity samples, which were the urine and lung, were evaluated on a Capintec™ CRC-15 dose calibrator.

Evaluation of the data indicates 76% of the injected Lu-177 remained in the lung at 9 days post injection. About 15% was excreted in the feces/urine. The rat skeleton (Bone) had 3.6%, and liver about 0.4%. Less than 1% of the injected radioactivity was found in any other organ or tissue.

### Example 6

### High pH Ho-166 Administration

Holmium-166 (Ho-166) was obtained from MURR. The solution was 52.4 mCi in 350 μL for a specific activity of 0.15 mCi/μL in 0.1 M HCl. The Ho-166 solution (10 μL) was placed in a vial and 5 μL of 0.1M NaOH was added to form the composition. The pH was measured with pH paper showing a pH of about 10.

A miniature drill was used to create a hole in the femur of an anesthetized Sprague Dawley rat. A miniature pump was used to deliver 3 μL of this Ho-166 composition into the hole created by the drill.

Two hours after the injection of the dose the rat was sacrificed and dissected. Tissues/organs excised and counted included bone (opposite femur), liver, kidneys, spleen, muscle, blood, heart, lung, pancreas as well as the injected femur. Counting was done by the use of a NaI gamma detector to determine the presence of radioactivity.

The amount of activity found in the site of injection was 92% of the injected dose. Less than 2% of the dose was found in the liver or in the rest of the bone. Total skeletal dose was determined by multiplying the % dose in the opposite femur by 25. No urine activity was evident.

### Example C (Comparative)

### Low pH Ho-166 Administered to Bone

Ho-166 in 0.1M HCl was obtained from MURR. The pH was measured with pH paper showing a pH of about 1. The miniature drill described above in Example 6 was used to create a hole in the femur of an anesthetized Sprague Dawley rat. The miniature pump described above was used to deliver 3 μL of Ho-166 solution into the hole created by the drill. Two hours after the injection of the dose the rat was sacrificed and dissected. The amount of activity found in the site of injection was 5% of the injected dose. However 52% of the dose was found in the liver and 23% of the dose was found in the rest of the bone. Total skeletal dose was determined by multiplying the % dose in the opposite femur by 25. The high amount of the dose found in non-target areas shows that this form of Ho-166 is not an effective way to dose patients.

### Example D (Comparative)

### Lu-177 pH 4.0-4.5

Lu-177 was received from PerkinElmer in 0.05M HCl. A solution was prepared containing Lu-177 in physiological saline. The final pH was adjusted to be between pH 4.0-4.5.

A BALB/c mouse was injected in the muscle mass of the left hind leg with 5 μL of the Lu-177 product using a 3/10 CC insulin syringe.

Gamma images were taken at various time points. After three days the images showed that the activity was spread across the whole body with very little remaining at the site of injection. A region of interest was drawn around the whole body and a second on the injection site using NucLear Mac Software by Scientific Imaging, Inc. version 5.9.5. The number of counts in the injection site and the whole body showed less than five percent of the activity remaining in the body was at the site of injection.

### Example E (Comparative)

### Lu-177 pH 4.0-4.5 Increased Volume

Lu-177 was received from PerkinElmer in 0.05M HCl at 19.74 Ci/mg. A solution was prepared containing 1000 μg of stable isotope as lutetium chloride/mL dissolved in physiological saline. 1704 of the solution was combined with 5 μl, of Lu-177. The final pH was determined to be between pH 4.0-4.5. Activity was determined with a Capintec™ CRC-15 dose calibrator to be 330 μCi.

A BALB/c mouse was injected in the muscle mass of the left hind leg with 50 μL of the Lu-177 product. A second BALB/c mouse was injected in the same manner with 100 μL of the Lu-177 solution. A 3/10 CC insulin syringe was used for both injections.

Gamma images were taken of both mice at various time points. A region of interest was set around the injection site and counts were determined using NucLear Mac Software by Scientific Imaging, Inc version 5.9.5. Background was subtracted and the counts were decay corrected. The amount of activity remaining at the site of injections is shown in Table 2.

TABLE 2
Retention at site of injection using Lu-177 pH 4.0-4.5 Increased Volume

| | Mouse | Time | Background | Decay | % Activity |
|---|---|---|---|---|---|
| | Injection | Passed | Corrected | Corrected | Remaining |
| Mouse # | Size (uL) | (Days) | Counts | Counts | at Site |
| 1 | 50 | 0.00 | 23443 | 23443 | 100% |
| 1 | 50 | 0.83 | 20566 | 22411 | 96% |
| 1 | 50 | 1.89 | 16489 | 20034 | 85% |
| 1 | 50 | 2.88 | 14401 | 19398 | 83% |
| 1 | 50 | 5.87 | 9823 | 18002 | 77% |
| 1 | 50 | 6.84 | 8647 | 17521 | 75% |
| 1 | 50 | 8.05 | 7452 | 17119 | 73% |
| 2 | 100 | 0.00 | 44173 | 44173 | 100% |
| 2 | 100 | 0.81 | 38738 | 42129 | 95% |
| 2 | 100 | 1.87 | 31517 | 38221 | 87% |
| 2 | 100 | 2.87 | 26747 | 35958 | 81% |
| 2 | 100 | 5.85 | 19060 | 34883 | 79% |
| 2 | 100 | 6.81 | 16010 | 32344 | 73% |
| 2 | 100 | 8.02 | 13430 | 30760 | 70% |

### Example F (Comparative)

### Sm-153-DOTMP

Sm-153 in 0.1 M HCl was obtained from MURR. The complex formed between Sm-153 and DOTMP was prepared by combining 5 μL of Sm-153 with 5.6 μL of a solution containing 13 mg/mL of DOTMP (previously adjusted to pH 7-8) and 4 μl, of water. An additional 5 μL of DOTMP solution was added to obtain high complex yields. The amount of Sm found as a complex was 99% by ion exchange chromatography. DOTMP was prepared and purified by known synthetic techniques. The chelant was greater than 99% pure.

The miniature drill described in Example 6 was used to create a hole in the femur of an anesthetized Sprague Dawley rat. The miniature pump described in Example 6 was used to deliver 2 μL of Sm-153-DOTMP solution into the hole created by the drill. Two hours after the injection of the dose the rat was sacrificed and dissected. The amount of activity found in the site of injection was 9% of the injected dose. None of the radioactivity was found in the liver and about 20% was found in the rest of the bone. Total skeletal dose was determined by multiplying the % dose in the opposite femur by 25. An average of 65% of the injected dose was found in the urine.

### Example 7

### Separation of Insoluble Particles

Y-90 is obtained from PerkinElmer in a minimal volume of 0.05M HCl solution. A NaOH solution is added to obtain a pH greater than about 10 resulting in the precipitation of insoluble particles. The mixture is centrifuged and the supernatant is removed. The remaining insoluble particles are re-suspended in saline. The resulting saline suspension is useful for the treatment of non-intracavitary undesirable tissue masses.

### Example 8

### Treatment of Canine osteosarcoma (I)

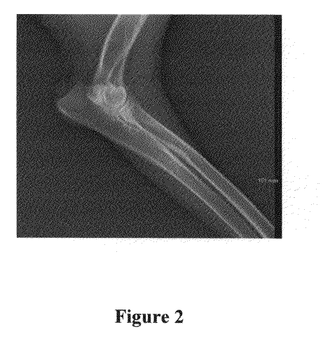

A 137 lb female Great Dane, 8½ years old, was presented in pain and limping. The dog was scanned with both X-ray and F-18 FDG PET/CT and the disease was determined to be isolated to a small, 2 cc osteosarcoma in the left proximal radius as shown in FIG. 2.

On the day of treatment, the dog was anesthetized and the proximal radius was surgically exposed. The osteosarcoma was easily discernable.

To a vial containing 10 mCi of Y-90 was added 100 μL of a 1.0 N NaOH solution using a pipette. The pipette was used to thoroughly mix the components. The resulting mixture contained 100 μCi/μL.

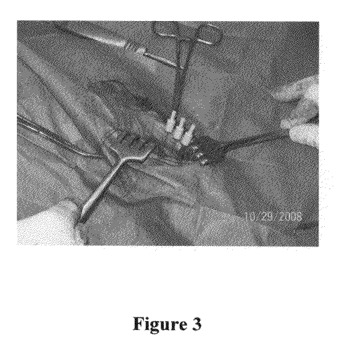

Using custom-made three piece adapters (hypodermic, cortex, stylet) and micro bone drill (Valco Instruments Company), three 0.45 mm holes, 1 cm apart, were drilled into the tumor. The hypodermic adapter anchors to bone and becomes the guide for the wire drill bit, the cortex adapter slides through the hypodermic adapter and extends into the hole drilled to avoid locational loss, the stylet adapter slides through the cortex adapter to prevent loss of body fluids and coagulation. FIG. 3 shows the use of this three piece adapter system.

One at a time using a 10 microliter syringe, 2 μL of the Y-90 composition (200 μCi) was injected into the tumor through each cortex adapter after removal of the stylet adapter. A total of 600 μCi was injected. Dosimetry calculations indicated that this quantity of composition, spaced as indicated, delivered a minimum of 50 Gy to the entire tumor mass. The adapters were removed immediately after injection. The incision was then closed and sutured and the surgical site stapled and dressed.

Scanning with a survey meter indicated all of the activity was localized in the tumor with very little detected in the bladder. Over the next several hours, slight activity was detected in the urine.

Pain relief was prompt—the following day the dog was walking without a limp. Normal survival time for a dog presenting with osteosarcoma is 6-9 months with the standard of care being amputation and chemotherapy. This dog lived for 9 months without these treatments and finally died of Dilated Cardiomyopathy, a disease common in Great Danes and unrelated to cancer. The dog was cancer free at time of death.

### Example 9

### Treatment of Canine osteosarcoma (II)

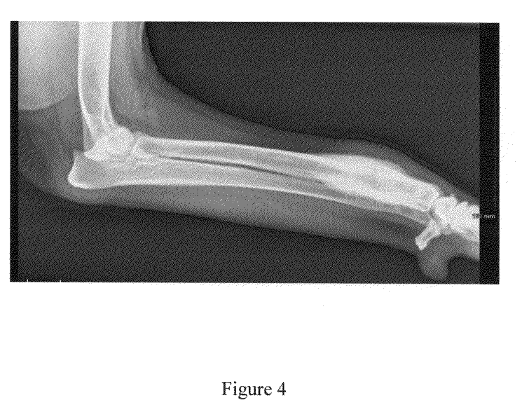

A 5¾ year old female Saint Bernard, 175 lbs, was presented in pain and limping. The dog was scanned with both X-ray and F-18 FDG PET/CT and the disease was determined to be isolated to a large, 67 cc osteosarcoma in the left distal radius as shown in FIG. 4.

On the day of treatment, the dog was anesthetized and the distal radius was surgically exposed. The osteosarcoma was easily discernable.

To a vial containing 18.5 mCi of Y-90 was added 185 μL of a 1.0 N NaOH solution using a pipette. The pipette was used to thoroughly mix the components.

The resulting composition contained 100 μCi/μL.

Using the three piece adapters and micro bone drill described in Example 7, thirty three 0.45 mm holes, 1 cm apart, were drilled into the tumor.

One at a time, using a 25 microliter syringe, 4 μL of the Y-90 composition (400 μCi) was injected into the tumor in two portions (deep and shallow) through each cortex adapter after removal of the stylet adapter. A total of 13.2 mCi was injected. Dosimetry calculations indicated that this quantity of composition, spaced as indicated, delivered a minimum of 50 Gy to the entire tumor mass. The adapters were removed immediately after injection. The incision was then closed and sutured and the surgical site stapled and dressed.

Scanning with a survey meter indicated all of the activity was localized in the tumor with very little detected in the bladder. The dose rate at the surgical site was 15 mR/hr. Over the next several hours, some activity was detected in the urine, the dose rate was 0.8 mR/hr.

Pain relief was prompt—the following day the dog was walking without a limp. The dog is still currently disease free at one year post treatment.

### CONCLUSIONS

The examples above are illustrative of the present invention. When compositions of radioisotope(s) prepared as described herein are administered in small volume, the vast majority of the isotope remains at the site of administration, even 13 days (two half lives) post injection (e.g. Example 2), compared with a similar administration of radioisotopes at low pH where a significant portion of the radioactivity migrates away from the site of administration (e.g. Example B). When administration of isotopes are made directly into the bone as taught herein, a significantly higher percentage of radioactivity can be delivered to bone compared to I.V. administration of a bone-seeking radiopharmaceutical where only about 0.1% of the radioactivity is taken up by a bone tumor. This allows a much lower total amount of radioactivity to be administered to deliver a much greater radiation dose to the target tissue.

The use of the compositions of this invention show in some cases, greater than 90% of the radioactivity is at the desired site with little to no activity in non-target organs or tissues. As stated above, in addition to practically eliminating the dose to non-target tissues and organs, much less radioisotope is needed. Finally, since more activity can be delivered to the tumor, resolution of the tumor is possible. In comparing the tumor growth rate in Example 2 to that of Example B, a therapeutic effect was clearly demonstrated.

Although the invention and processes have been described with reference to these embodiments, those of ordinary skill in the art may, upon reading this application, appreciate changes and modifications which may be made which do not depart from the scope and spirit of this invention as described above or claimed hereafter.

What is claimed is:

1. A pharmaceutically-acceptable composition which comprises insoluble particles in a pharmaceutically-acceptable, aqueous medium, obtained by adding an alkaline material to an aqueous solution comprising a rare earth or rare earth type radionuclide or combination thereof, forming a suspension, slurry or emulsion, said composition having a pH greater than about 7, and wherein a therapeutically-effective quantity thereof is administered in one or more locations into or near a non-intracavitary undesirable tissue mass in one or more locations in an animal or human in need of such treatment, such that greater than about 75% of the administered quantity remains at the site of administration for at least two half lives of the radionuclide.

2. The pharmaceutically-acceptable composition of claim 1 which has the insoluble particles therein separated from its initial composition by filtering, centrifuging or decanting, and thereafter a therapeutically-effective quantity of the separated insoluble particles is re-suspended in a pharmaceutically-acceptable medium and administered as in claim 1.

3. The pharmaceutically-acceptable composition of claim 1 wherein the pH of the composition is from about 8 to about 14.

4. The pharmaceutically-acceptable composition of claim 3 wherein the pH of the composition is from about 8 to about 11.

5. The pharmaceutically-acceptable composition of claim 1 wherein the alkaline material is sodium hydroxide or potassium hydroxide.

6. The pharmaceutically-acceptable composition of claim 1 wherein the amount of the administered dose remaining at the administration site is greater than 90% after two half lives of the radionuclide.

7. The pharmaceutically-acceptable composition of claim 1 wherein the radionuclide is Sm-153, Ho-166, Y-90, Pm-149, Gd-159, Lu-177, Yb-175, Pb-212, Bi-212, Bi-213, or Ac-225.

8. The pharmaceutically-acceptable composition of claim 7 wherein the radionuclide is Sm-153, Ho-166, Y-90, Bi-212, Bi-213, Ac-225, or Lu-177.

9. The pharmaceutically-acceptable composition of claim 7 wherein the composition is contained in a volume of less than about 50 microliters per delivery site as a therapeutically-effective radiation dose in an undesirable tissue mass.

10. The pharmaceutically-acceptable composition of claim 9 wherein the volume is less than about 20 μL.

11. The pharmaceutically-acceptable composition of claim 10 wherein the volume is less than about 10 μL.

12. The pharmaceutically-acceptable composition of claim 11 wherein the volume is less than about 2 μL.

13. The pharmaceutically-acceptable composition of claim 9 wherein the composition is deposited in multiple locations within the undesirable tissue mass such that an effective therapeutic radiation dose is delivered to the entire tissue mass.

14. A method for the therapeutic treatment of a non-intracavitary undesirable tissue mass in an animal or human in need of such treatment, wherein a pharmaceutically-acceptable composition of claim 1 or 2 is administered in a therapeutically-effective dose of claim 9 or 13.

15. The method of claim 14 wherein the composition is as defined as in claim 6.

16. The method of claim 14 wherein the composition is as defined as in claim 8.

17. The method of claim 14 wherein the composition is deposited in multiple locations within the undesirable tissue mass such that an effective therapeutic radiation dose is delivered to the entire tissue mass.

18. The method of claim 14 wherein the undesirable tissue mass is a cancerous mass.

19. The method of claim 18 wherein the cancer is located in bone, prostate, liver, lung, brain, muscle, breast, cervix or skin.

20. The method of claim 19 wherein the cancer is bone and a miniature drill is used to create a hole or multiple holes in the bone by which a needle or catheter can be inserted through the hole(s) and a device capable of delivering small volumes of fluid is used to deliver the dose.

21. The method of claim 20 wherein the dose is delivered via a pump or syringe.

22. The method of claim 14 wherein the placement of the composition is guided by an imaging technique.

23. The method of claim 22 wherein the imaging technique is PET, CT, ultrasound, fluoroscopy, or MRI.
